(19) United States
(12) Patent Application Publication
(10) Pub. No.: US 2007/0060873 A1
(43) Pub. Date: Mar. 15, 2007

(54) Milking apparatus

(76) Inventors: Katsuyuki Hiraoka, Osaka-fu (JP); Tomoyuki Oura, Osaka-fu (JP); Yasuo Morifuji, Osaka-fu (JP)

Correspondence Address: ARENT FOX PLLC, 1050 CONNECTICUT AVENUE, N.W., SUITE 400, WASHINGTON, DC, 20036, US

(21) Appl. No.: 11/226,445

(22) Filed: Sep. 15, 2005

(51) Int. Cl. A61M 1/06

(52) U.S. Cl. 604/74

(57) ABSTRACT

Disclosed is a milking apparatus that prevents extracted mother's milk from flowing into a pump, thereby keeping the mother's milk hygienic. A first space in a milking head communicating with a milking cup and a second space communicating with a space in a rubber bulb are separated from each other by a partition film, and air in the rubber bulb is exchanged solely between the interior of the rubber bulb and the second space, which is surrounded by the partition film and an upper cover portion of the milking head.

### BACKGROUND OF THE INVENTION

The present invention relates to a milking apparatus for extracting mother's milk.

As shown in FIG. 6, in a conventional milking apparatus 51, a milking head 54 is mounted on an upper portion of a bottle 52 for storing extracted milk by threaded engagement through intermediation of a partition member 53, and a rubber bulb 55 is mounted to the milking head 54 (see, for example, Japanese Utility Model Application Laid-Open S52-15896). In FIG. 6, reference numeral 56 denotes a milking cup, and reference numeral 57 denotes a check valve.

An operation of the milking apparatus 51 will be described. First, when the rubber bulb 55 is compressed, with the milking cup 56 pressed against the breast, air forced out of the rubber bulb 55 flows into the milking head 54. The air that has flowed into the milking head 54 pushes open the check valve 57, and flows into the bottle 52. At this time, the air flowing into the bottle 52 is discharged to the outer space through a slight gap in the threaded engagement portion between the bottle 52 and the milking head 54. When, thereafter, the rubber bulb 55 is released from the compressed state, the air in the milking head 54 is sucked into the rubber bulb 55. In this state, the air in the bottle 52 does not flow into the milking head 54 due to the check valve 57, so that negative pressure is created in the hermetically sealed milking head 54, thereby effecting milking. Next, the rubber bulb 55 is compressed. Then, the extracted mother's milk pushes open the check valve 57 together with the air, and flows into the bottle 52.

In the above-described milking apparatus, the air forced out of and sucked into the rubber bulb flows into and out of the mother's milk inflow space extending from the milking cup to the bottle, so that the extracted mother's milk may enter the pump in the form, for example, of droplets or may flow into the pump when the bottle topples over. Further, even if no mother's milk flows into the pump, the odor, for example, of the rubber bulb forming the pump may be allowed to be mixed into the air in the pump. When the air mixed with the odor flows into the milking head, the odor may permeate into the mother's milk.

### SUMMARY OF THE INVENTION

It is accordingly an object of the present invention to provide a milking apparatus in which the above-mentioned problems have been solved.

In order to attain the object described above, a milking apparatus according to the present invention includes a bottle, a milking head mounted to an opening of the bottle and having a milking cup flared out like a trumpet, and a negative pressure generating unit mounted to the milking head and generating negative pressure inside the milking cup, characterized in that the negative pressure generating unit is equipped with a partition film arranged inside the milking head and dividing the space in the milking head into a first space including the interior of the milking cup and communicating with the bottle and a second space, and a discharging device for discharging a fluid filling the second space.

According to the present invention, of the first space and the second space separated from each other by the partition film, the second space is contracted through the discharging effect of the discharging device provided in the negative pressure generating unit to move the partition film to the second space side, thereby expanding the first space communicating with the milking cup to generate negative pressure in the first space. In this way, according to the present invention, the space inside the milking head is divided by the partition film into the first space communicating with the milking cup and the second space capable of discharging the fluid filling its interior by means of the discharging device, so that it is possible to prevent the extracted mother's milk from flowing, for example, into the pump through the second space. Further, according to the present invention, the air in the first space and the fluid in the second space are not mixed with each other, so that even when, for example, a pump is provided as the discharging device, no intake/exhaust air from the pump is mixed with the air in the mother's milk inflow space extending from the milking cup to the bottle. Thus, it is possible to prevent the air in the pump from coming into contact with the extracted mother's milk. The partition film may be formed, for example, of a thin-film-like elastic member, or of a hollow expansion/contraction member equipped with a bellows portion. Further, as stated above, the “fluid” filling the second space is not mixed with the air in the first space, so that it is possible to use various fluids as this fluid, for example, gases other than air, such as nitrogen gas, or liquids, such as water.

The discharging device may be formed, for example, by a hollow elastic member adapted to suck in the fluid in the second space through elastic restoration after compression. In this construction, it is possible to discharge the fluid into the second space by compressing the hollow elastic member, and it is possible to suck the fluid into the hollow elastic member from the second space by canceling the compressed state to effect elastic restoration to the former configuration. In particular, due to the discharging effect accompanying the compressive deformation of the hollow elastic member, the partition film is moved to the first space side by sending the fluid into the second space, and the air in the first space is forcibly sent to the milking cup side from the partition film side. Thus, by repeating the compression/releasing of the hollow elastic member forming the discharging device, it is possible to push in as well as suck the breast, so that, as compared with the conventional device, which simply sucks and releases the breast, it is possible to obtain a superior massaging effect.

In addition to the above-mentioned construction of the milking apparatus, a massage effect during milking can also be attained by providing: a milking cup capable of generating negative pressure inside; and a pad formed of an elastic material and attached to an opening of the milking cup, characterized in that the pad is provided with a thick-walled portion which is formed more thick-walled than the rest of the pad and which protrudes on the side thereof opposite to the breast when left alone and comes into contact with an inner peripheral surface of the milking cup upon generation of negative pressure inside the milking cup to thereby protrude on the breast side. That is, when left alone (when the interior of the milking cup is at normal pressure), the thick-walled portion provided on the pad protrudes on the side opposite to the breast, so that the user can attach the pad to the breast without feeling any discomfort. In the negative pressure state (the state in which the pad is sucked into the milking cup), the thick-walled portion has contact with the inner surface of the milking cup to protrude on the breast side, so that the breast is stimulated. Thus, by repeatedly switching the interior of the second space 21 between the negative pressure state and the normal pressure state, it is possible to obtain a remarkable massaging effect.

Apart from the above-mentioned hollow elastic member, it is also possible for the discharging device to be formed by an electric pump. The electric pump is equipped, for example, with a power source, a motor driven through power supply from the power source, and a pump space adapted to expand and contract as the motor is driven. The electric pump may be constructed such that in the state in which the pump space is expanded, air is drawn into the pump space from the second space, and that in the state in which the pump space is contracted, the fluid in the pump space (which mainly consists of air) is released to the exterior.

As stated above, various devices can be used as the discharging device. In the case, for example, of the construction using the hollow elastic member as described above, in which a fixed amount of fluid moves between the second space and the interior of the discharging device, it matters to what extent the first space is to be expanded. In view of this, in the present invention, an opening/closing mechanism for opening or closing the second space with respect to the exterior is provided in the milking apparatus. In this case, air is preferably used as the fluid filling the second space. In this construction, the opening/closing mechanism is previously placed in the opening state to allow the second space to communicate with the exterior, and, in this state, the hollow elastic member is compressed to release the air in the second space and in the hollow elastic member to the exterior. Thereafter, the opening/closing mechanism is placed in the closing state to close the second space with respect to the exterior, and the compressed state of the hollow elastic member is canceled, whereby the air in the second space flows into the hollow elastic member. As a result, it is possible to place the second space in a negative pressure state, and to move the partition film greatly to the second space side, so that the expansion volume of the first space is further enlarged, making it possible to generate a very high negative pressure in the first space. Further, when the second space is placed in a negative pressure state, the interior of the hollow elastic member is also placed in a negative pressure state, so that the user can compress the hollow elastic member with a very small force to perform milking operation. When the discharging device is formed by an electric pump, this opening/closing mechanism functions as a normal pressure restoration device for restoring the second space from the negative pressure state to the normal pressure state.

Various types of mechanism can be used as the opening/closing mechanism. For example, it is also possible to adopt a construction in which the second space is opened to the exterior through elastic deformation of the elastic member forming the opening/closing mechanism by operation from outside.

Further, it is possible to provide a detecting portion for detecting displacement of the partition film, and to perform the opening/closing operation of the opening/closing mechanism automatically according to the displacement amount of the partition film detected by the detecting portion. Alternatively, it is also possible to provide a detecting portion for detecting the pressure of the first space, performing the opening/closing operation automatically according to the pressure of the first space detected by the detecting portion.

In this construction, when the pressure of the first space is reduced to a level not higher than a predetermined pressure, external air is automatically drawn into the second space, and when a certain amount of external air is drawn into the second space, that is, when the pressure of the first space exceeds a predetermined pressure value, the communication between the second space and the exterior is automatically canceled, and the operation of bleeding the air in the second space is repeatedly performed with a fixed rhythm. In this way, a so-called “pulsation effect” can be obtained by switching the pressure condition of the first space with a fixed rhythm, so that it is possible to massage the breast effectively, thereby further facilitating the milking operation.

As stated above, the present invention is characterized in that the first space, which includes the milking cup and communicates with the bottle, is expanded to thereby generate negative pressure in the space. Thus, to generate negative pressure more effectively, it is necessary to make the volume of the first space prior to expansion (with the first space and the second space in the same pressure condition) as small as possible. For this purpose, according to the present invention, the air in the first space is permitted to flow into the bottle, and, at the same time, there is provided between the first space and the bottle a check valve for preventing the air in the bottle from flowing into the first space. Due to this arrangement, the first space and the space in the bottle are separated from each other, and it is possible to reduce the volume of the first space generating negative pressure.

In the milking apparatus of the present invention, the extracted mother's milk and the air in the pump are prevented from coming into contact with each other, and the extracted mother's milk is prevented from flowing into the pump, thereby making it possible to keep the mother's milk clean from the hygienic viewpoint.

### BRIEF DESCRIPTION OF THE DRAWINGS

FIG. 1 is a longitudinal sectional view of a milking apparatus according to a first embodiment of the present invention;

FIG. 2 is a longitudinal sectional view of a modification of the first embodiment;

FIG. 3 is a longitudinal sectional view of a milking apparatus according to a second embodiment of the present invention;

FIG. 4 is a longitudinal sectional view of a modification of the second embodiment;

FIGS. 5A and 5B are enlarged partial sectional views of a milking cup; and

FIG. 6 is a longitudinal sectional view of a conventional milking apparatus.

### DESCRIPTION OF THE PREFERRED EMBODIMENTS

In the following, embodiments of the present invention will be described with reference to the drawings. In the following description of the embodiments, the members and portions having the same functions are indicated by the same reference numerals.

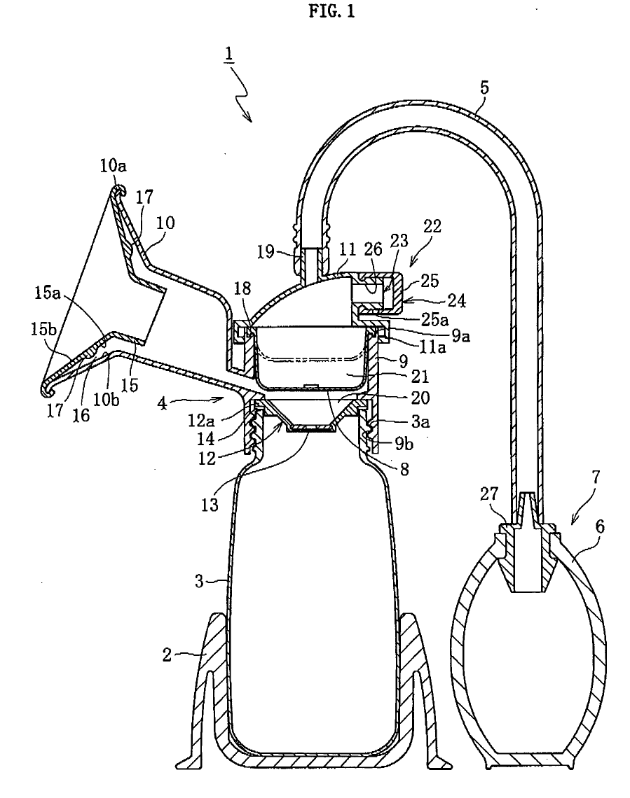

FIG. 1 is a longitudinal sectional view of a milking apparatus 1 according to the first embodiment of the present invention. The milking apparatus 1 is mainly composed of a bottle 3 held upright by a pedestal 2, a milking head 4 mounted to the bottle 3, a discharging device 7 formed by a tube 5 and a rubber bulb (hollow elastic member) 6 mounted to the milking head 4 through the intermediation of the tube 5, and a partition film 8 provided in the milking head 4.

The milking head 4 is equipped with a substantially cylindrical base portion 9, a milking cup 10 extending outwardly from the outer periphery of the base portion 9, and an upper cover portion 11 mounted to an upper open end 9a of the base portion 9. The base portion 9 is mounted to the bottle 3 through the intermediation of a partition member 12 by threadedly engaging a screw portion 9b formed in the inner periphery of the lower portion thereof with a screw portion 3a formed in the outer periphery of the upper end portion of the bottle 3. The partition member 12, provided between the bottle 3 and the milking head 4, is equipped with a check valve 13, and a radial groove 14 is formed in the lower surface of a flange portion 12a of the partition member 12.

The milking cup 10 has a portion flared in a trumpet-like fashion toward the outer side of the base portion 9, and is formed integrally with the base portion 9, with its inner space communicating with the inner space of the base portion 9. A pad 15 is detachably attached to an opening 10a of the milking cup 10. The pad 15 is formed of an elastic material, such as silicone rubber or polypropylene, and, in the state in which the pad 15 is mounted to the milking cup 10, there is formed a gap 16 between an outer peripheral surface (the surface on the side opposite to the breast) 15a of the pad 15 and an inner peripheral surface 10b of the milking cup 10. The pad 15 functions as an adapter for fitting the breast or nipple of each individual to the milking cup according to its size, and, in the state in which the nipple is sucked into the milking head 4, the pad 15 as a whole is sucked into the milking head by an amount corresponding to the gap 16, whereby not only the nipple, but also the breast in intimate contact with the pad 15 can be sucked into the milking head 4 over a wide range. Thus, through attachment of the pad 15 to the breast, the user can perform milking without feeling any pain.

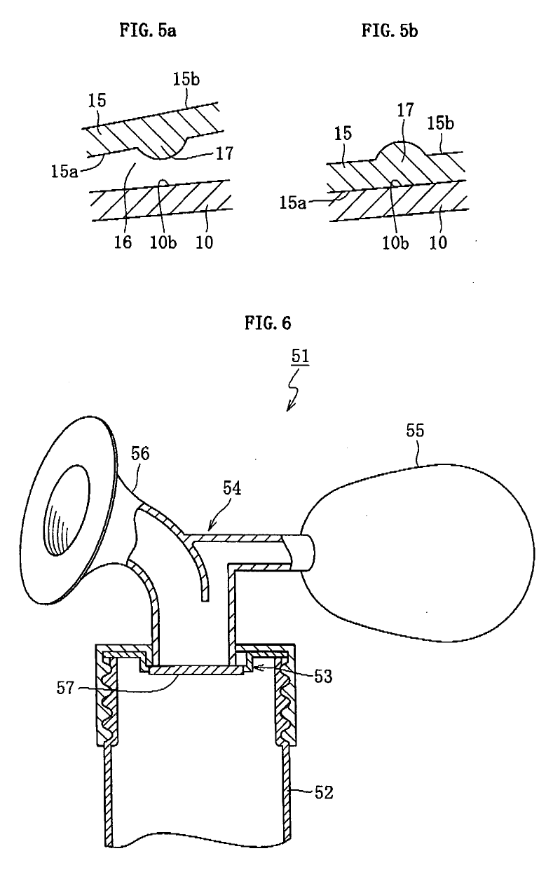

Further, the pad 15 is provided with a thick-walled portion 17 that is more thick-walled than the rest of the pad. As shown, for example, in FIG. 5A, when the inner space of the milking cup 10 is under normal pressure, the thick-walled portion 17 protrudes on the outer peripheral surface (the surface on the side opposite to the breast) 15a side of the pad 15. When the inner space of the milking cup 10 is placed under negative pressure, the pad 15 is sucked into the milking head 4, and the thick-walled portion 17 protruding from the outer peripheral surface 15a abuts the inner peripheral surface 10b of the milking cup 10. As shown in FIG. 5B, when, in this state, the pad 15 is further pulled toward the interior of the milking head 4 by an amount corresponding to the gap 16, the outer peripheral surface 15a of the pad 15 and the inner peripheral surface 10b of the milking cup 10 are brought into intimate contact with each other, and the thick-walled portion 17 is pushed within the pad 15 to protrude on the inner peripheral surface (the breast side surface) 15b side. In this way, when the pad 15 is attached to the breast, the inner peripheral surface 15b of the pad 15 becomes smooth, so that the user can attach the pad 15 without feeling any discomfort. In the state in which the inner space of the milking cup 10 is under negative pressure, the thick-walled portion 17 protrudes on the inner peripheral surface (the breast side surface) 15b side of the pad 15 which is in intimate contact with the breast, so that the breast is stimulated. Thus, by alternately switching the pressure state of the interior of the milking cup 10 between normal pressure and negative pressure, a kind of massage effect is obtained, making it possible to perform milking more effectively. While in this embodiment a plurality of thick-walled portions 17 are provided over the circumference of the pad 15, this should not be construed restrictively. It is also possible to form the thick-walled portion 17 in an annular configuration over the entire periphery of the pad 15.

The upper cover portion 11 is fixed to the base portion 9 by fitting the lower end portion 11a thereof into the upper open end 9a, with a partition film 8 being nipped between the lower end portion 11a and the upper opening end 9a of the base portion 9. Formed at the vertex of the upper cover portion 11 is an upper opening 19 directed to the side opposite to the bottle 3, and the rubber bulb 6 is mounted to the upper opening 19 through the intermediation of the tube 5. While in this embodiment the upper cover portion 11 is formed in a substantially dome shape with the vertex offset outwardly, it is also possible for the upper cover portion 11 to be in, for example, a semi-spherical configuration with its vertex at the center. Apart from this, it can assume various configurations.

As shown in FIG. 1, in this embodiment, the partition film 8 is a thin-film-like elastic member formed as a bottomed cylinder. As the material of the partition film 8, there is used, for example, silicone rubber, polypropylene or the like, which is flexible and odorless. The partition film 8 is fixed in position within the milking head 4 by press-fitting a flange portion 18 formed in the peripheral edge of the partition film 8 into the upper open end 9a of the base portion 9 and nipping it between the upper cover portion 11 connected to the tube 5 and the milking head 4. As a result, the space in the milking head 4 is divided into a first space 20 including the inner space of the milking cup 10 and a second space 21 communicating solely with the rubber bulb 6 through the tube 5.

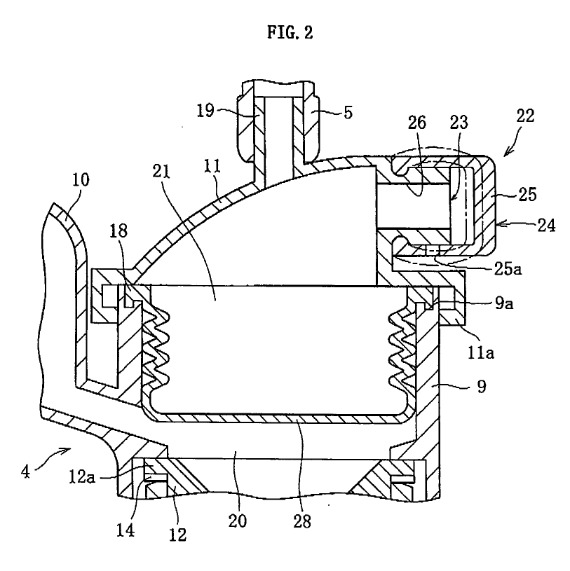

The milking head 4 is equipped with an opening/closing mechanism 22 for opening or closing the second space 21 to the external space. As shown, for example, in FIG. 2, in this embodiment, the opening/closing mechanism 22 is formed by a side opening 23 formed in the outer periphery of the upper cover portion 11 and directed outwardly and an elastic member 24 attached to the side opening 23 and adapted to open the second space 21 to the exterior through elastic deformation by operation from outside. The elastic member 24 is formed by a rubber cap 25 formed, for example, as a bottomed cylinder and having a through-hole 25a in its side surface, and is mounted to the side opening 23 so as to cover a hole 26 communicating with the second space 21. In the state in which the rubber cap 25 attached to the side opening 23 is inwardly pressed, a gap is formed between the inner periphery of the rubber cap 25 and the outer periphery of the side opening 23 (as indicated, for example, by the alternate long and short dashed lines in FIG. 2). The second space 21 is opened to the external space through the through-hole 25a of the rubber cap 25 and the hole 26 facing this gap. Thus, when the air pressure in the second space 21 is lower than the atmospheric pressure, external air flows into the second space 21. Although not shown, apart from the above-described construction, the opening/closing mechanism 22 can, for example, also be formed by a pushbutton and a valve communicating with the hole 26 by opening and closing upon manipulation of the pushbutton. Alternatively, it may be formed by a cover rotatably mounted to the side opening 23 equipped with the hole 26 and adapted to adjust through rotation the state of communication (the amount of air flowing in or out) between the exterior and the hole 26.

Next, the operation of the milking apparatus 1 will be described.

When the rubber bulb 6 is compressed, with the rubber cap 25 depressed, air forced out of the rubber bulb 6 flows into the second space 21 through the tube 5 into the second space 21 surrounded by the partition film 8 and the upper cover portion 11, and is further discharged to the exterior from the hole 26 through the through-hole 25a of the rubber cap 25. In this state, the partition film 8 is in a neutral position (the position indicated by the solid line in FIG. 1) as shown in FIG. 1. After this, the rubber cap 25 is released from the depressed state to attain a condition in which the second space 21 is closed to the external space, and then the rubber bulb 6 is released from the compressed state. As a result, the air inside the second space 21 flows into the space in the rubber bulb 6, and the second space 21 is placed under negative pressure. Further, the partition film 8 moves to the second place 21 side (the position indicated by the chain double-dashed line in FIG. 2) to contract the second space 21. As a result, the first space 20 expands by an amount corresponding to the contraction of the second space 21, and is changed to a negative pressure state, so that the breast previously pressed against the milking cup 10 (i.e., in intimate contact with the pad 15 mounted to the milking cup 10) is sucked into the first space 20 together with the nipple, and mother's milk is extracted.

Next, when the rubber bulb 6 is compressed, air forced out of the rubber bulb 6 flows into the second space 21 through the tube 5, and causes the partition film 8 to move to the first space 20 side. As a result, the air in the compressed first space 20 and the extracted mother's milk push open the check valve 13 of the partition member 12 provided between the bottle 3 and the milking head 4, and flow into the bottle 3. In this way, compression of the rubber bulb 6 and canceling of the compression thereof are repeated, whereby extracted mother's milk is stored in the bottle 3. The air flowing into the bottle 3 together with the mother's milk passes through the radial groove 14 provided in the lower surface of the flange portion 12a of the partition member 12, and is further discharged to the external space through a slight gap of the threaded engagement portion between the bottle 3 and the milking head 4. Thus, normal pressure is constantly maintained in the bottle 3. The gap in the threaded engagement portion between the bottle 3 and the milking head 4 is open so as to be watertight although not airtight. Thus, even if the bottle 3 topples over, there is no fear of the mother's milk in the bottle 3 flowing out through the threaded engagement portion.

In this way, the partition film 8 is provided in the milking head 4 to divide the space in the milking head 4 into the first space 20 including the milking cup 10 and the second space 21 communicating with the rubber bulb 6 through the tube 5, whereby the space in the milking cup 10 and the space in the rubber bulb 6 are separated from each other. Thus, it is possible to prevent the extracted mother's milk from flowing into the rubber bulb 6 and to keep the mother's milk hygienic. Further, the air sent from the rubber bulb 6 to the second space 21 does not flow into the inflow route (space) for mother's milk extending from the milking cup 10 to the bottle 3 by way of the first space 20, so that it is possible to prevent the air in the rubber bulb 6 from coming into contact with the extracted mother's milk.

Further, due to the provision of the partition member 12 between the bottle 3 and the milking head 4, the interior of the bottle 3 and the interior of the milking head 4 are separated from each other, and the volume in the neutral state of the first space generating negative pressure is reduced. As a result, the expansion ratio with respect to the original volume of the first space 20 (the volume thereof under the same pressure state as the second space 21) is increased, making it possible to generate a still higher negative pressure in the first space 20. Further, the opening/closing mechanism 22 for opening or closing the second space 21 to the external space is provided in the milking head 4, so that, by operating the opening/closing mechanism 22 and the rubber bulb 6 in the above order, the second space 21 is placed under negative pressure, making it possible to increase the expansion volume of the first space 20. Further, by placing the second space 21 and the rubber bulb 6 under negative pressure, it is possible to compress the rubber bulb 6 with small force, so that it is possible to perform milking while relieving the physical burden on the user. While in the example shown the opening/closing mechanism 22 is provided in the upper cover portion 11 of the milking head 4, this should not be construed restrictively. As long as it can open or close the second space 21 to the external space, the opening/closing mechanism 22 may also provided at some other position, for example, at the mounting portion 27 between the rubber bulb 6 and the tube 5.

Further, by sending air into the second space 21 by compressing the rubber bulb 6, the partition film 8 greatly expands to the first space 20 side, and the air in the first space 20 is pushed in toward the partition member 12 and pushed in toward the milking cup 10. As a result, the breast pressed against the milking cup 10 is pushed by the air pushed in toward the milking cup 10. In this way, according to the present invention, during milking, the breast is not only sucked but also repeatedly stimulated by pushing, etc., so that it is possible to obtain a remarkable massaging effect, making it possible to perform milking efficiently.

The milking apparatus of the present invention is not restricted to the above-described embodiment. For example, as shown in FIG. 2, the partition film 8 shown in FIG. 1 may also be formed as a hollow expansion/contraction member 28 equipped with a bellows portion. The expansion/contraction member 28 generates negative pressure in the milking head 4 by making expanding and contracting movements along the bellows portion. Thus, it is desirable that the material, thickness, inner volume, etc. of the expansion/contraction member 28 be determined appropriately to a degree that the negative pressure generated in the milking head 4 does not hinder the expanding and contracting movements of the expansion/contraction member 28. Apart from this, the discharging device 7 may also be formed as a device driven by an arbitrary drive source and including a cylinder as a component. In this case, although not shown, the piston surface of the cylinder forms a plane serving as a partition for the second space.

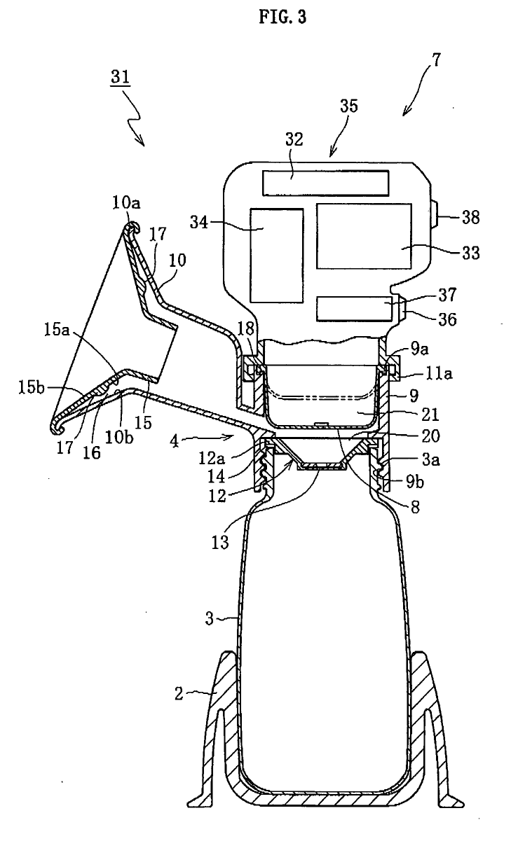

FIG. 3 is a longitudinal sectional view of a milking apparatus 31 according to the second embodiment of the present invention. While in the first embodiment the discharging device 7 is formed by the tube 5 and the rubber bulb 6 connected to the second space 21 through the intermediation of the tube 6, in the milking apparatus 31 of this embodiment, the discharging device 7 is formed by an electric pump 35 equipped with a power source 32, a motor 33 driven by power supply from the power source 32, and a pump space 34 that expands and contracts as the motor 33 is driven. Although not shown, the pump space 34 (for details, see, for example, JP2004-000486A) is formed between a diaphragm connected to the rotation shaft of the motor 33 and adapted to oscillate with the driving of the motor 33 and a diaphragm plate arranged in the pump 35 so as to be opposed to the diaphragm. For example, the diaphragm plate is equipped with a check valve for sucking air into the pump space 34 from the second space 21 in the state in which the pump space 34 is expanded, and a check valve for releasing the air in the pump space 34 to the exterior in the state in which the pump space 34 is contracted. In FIG. 3, reference numeral 36 indicates a pushbutton, reference numeral 37 indicates an opening/closing mechanism that opens the second space 21 to the exterior when the pushbutton 36 is depressed, and reference numeral 38 indicates a changeover switch for switching the motor 33 between the ON and OFF states.

Next, the operation of the milking apparatus 31 will be described.

Through manipulation of the changeover switch 38, the power source 32 and the motor 33 are electrically connected with each other, thereby driving the motor 33. With the driving of the motor 33, the diaphragm connected to the rotation shaft of the motor 33 oscillates, causing the pump space 34 formed between the diaphragm and the diaphragm plate to expand and contract. When the pump space 34 is expanded, air is drawn into the pump space 34 from the second space 21 by the check valve provided in the diaphragm plate, and when the pump space 34 is contracted, the air in the pump space 34 is released to the exterior by the check valve other than the check valve for drawing air into the pump space 34 from the second space 21. As a result, the air inside the second space 21 flows out to the exterior through pump 34, and the second space 21 is placed under negative pressure. Further, the partition film 8 moves to the second place 21 side (the position indicated by the chain double-dashed line in FIG. 3) to contract the second space 21. As a result, the first space 20 expands by an amount corresponding to the contraction of the second space 21, and is changed to a negative pressure state, so that the breast previously pressed against the milking cup 10 (i.e., in intimate contact with the pad 15 mounted to the milking cup 10) is sucked into the first space 20 together with the nipple, and mother's milk is extracted.

When communication is established between the second space 21 and the exterior through depression of the pushbutton 36 (through manual operation of the opening/closing mechanism 37), external air at normal pressure flows into the second space 21 in the negative pressure state, causing the partition film 8 to move to the first space 20 side. As a result, the air in the compressed first space 20 and the extracted mother's milk push open the check valve 13 of the partition member 12 provided between the bottle 3 and the milking head 4, and flow into the bottle 3. In this way, the second space 21 is repeatedly switched between negative pressure state and normal pressure state, whereby extracted mother's milk is stored in the bottle 3.

As stated above, when the electric pump 35 is used, the exhaust operation can be effected through switch manipulation, so that the physical burden on the user, such as the compression of the rubber bulb 6, is relieved, thus facilitating milking. Further, as in the first embodiment, the space in the milking cup 10 (the first space 20) and the space in the pump 35 capable of communicating with the exterior (the second space 21) are separated from each other by the partition film 8, so that the extracted mother's milk is prevented from flowing into the pump 35 or coming into contact with the air in the pump 35 or the external air, thereby making it possible to perform milking in a more hygienic state.

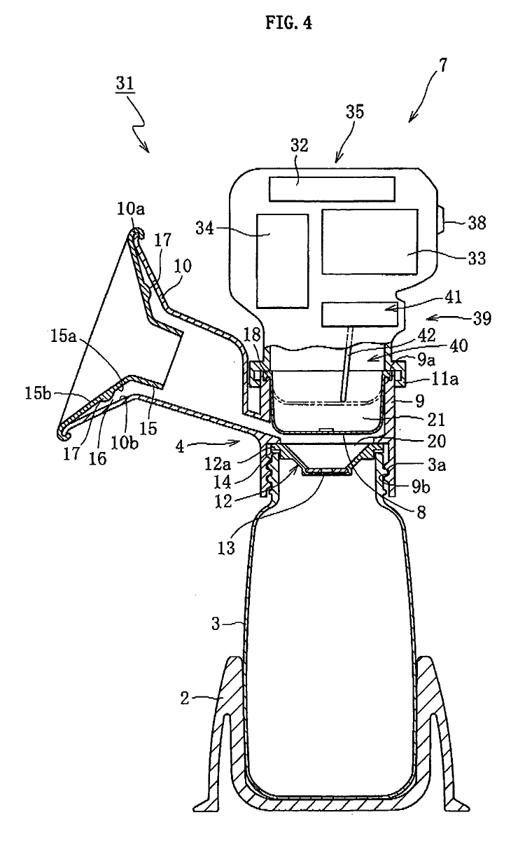

Further, while in this embodiment the user manipulates the opening/closing mechanism 37 (depresses the pushbutton 36) to thereby restore the second space 21 to normal pressure, it is also possible to provide a pressure adjusting device 39 which, when the pressure of the first space 20 is reduced to a predetermined pressure, automatically establishes communication between the second space 21 and the exterior to restore the pressure of the second space 21 to normal pressure or raise it to a predetermined pressure. The pressure adjusting device 39 is formed by a detecting portion 40 for detecting the pressure state of the first space 20, and an opening/closing mechanism 41 for changing the state of communication between the second space 21 and the exterior based on the detection result obtained by the detecting portion 40. The detecting portion 40 may, for example, be one that indirectly detects the pressure state of the first space 20 by detecting the position or displacement of the partition film 8 in the milking head 4. As shown, for example, in FIG. 4, in this embodiment, it is formed by a contact type detecting member 42 which detects the position or displacement of the partition film 8 by coming into contact with the partition film 8. In this case, when the detecting member 42 comes into contact with the partition film 8 that has moved to a predetermined position in the milking head 4 (the position indicated by the chain double-dashed lines in FIG. 4), communication is automatically established between the second space 21 and the exterior by the opening/closing mechanism 41, which is mechanically or electrically linked with the detecting member 42, and when the detecting member 42 is out of contact with the partition film 8, the communication between the second space 21 and the exterior is automatically canceled by the opening/closing mechanism 41. While in the example shown the detecting portion 40 is formed by the contact type detecting member 42 detecting the position of the partition film 8, this should not be construed restrictively. For example, although not shown, it is also possible to adopt a non-contact type detecting portion (including a displacement sensor) detecting the position or displacement of the partition film 8 in a non-contact fashion. Further, it is also possible to detect a reduction in the pressure of the first space 20 to a level below a predetermined pressure value by using a detecting portion (including a pressure sensor) which directly detects the pressure condition of the first space 20.

As stated above, with the pressure detecting device 39, it is possible to obtain a so-called “pulsating effect” causing the pressure of the first space 20 to be raised and lowered with a fixed rhythm, so that it is possible to massage the breast effectively with a fixed rhythm, making it easier to extract mother's milk. Further, the second space is automatically switched between open and closed states with respect to the exterior according to the pressure condition in the second space 21, so that it is possible to omit the manually operated opening/closing mechanism 37, making it possible to perform milking more easily. Of course, it is also possible to provide the manually operated opening/closing mechanism 37 as a standby device for emergency use separately from the pressure adjusting device 39.

What is claimed is:

1. A milking apparatus comprising: a bottle; a milking head mounted to an opening of the bottle and having a milking cup flared out like a trumpet; and a negative pressure generating unit mounted to the milking head, for generating negative pressure inside the milking cup,
wherein the negative pressure generating unit is equipped with an expandable partition film arranged inside the milking head and dividing the space in the milking head into a first space including the interior of the milking cup and communicating with the bottle and a second space, and a discharging device for discharging a fluid filling the second space.

2. A milking apparatus according to claim 1, wherein the discharging device is a hollow elastic member that sucks in the fluid in the second space through elastic restoration after compression.

3. A milking apparatus according to claim 1, wherein the discharging device is an electric pump.

4. A milking apparatus according to claim 1, further comprising an opening/closing mechanism for opening or closing the second space to an exterior.

5. A milking apparatus according to claim 4, wherein the second space is opened to the exterior by elastic deformation of an elastic member constituting the opening/closing mechanism through operation from outside.

6. A milking apparatus according to claim 4, further comprising a detecting portion for detecting a displacement of the partition film, wherein the opening/closing mechanism is switched between opening and closing according to the displacement amount of the partition film.

7. A milking apparatus according to claim 4, further comprising a detecting portion for detecting a pressure of the first space, wherein the opening/closing mechanism is switched between opening and closing according to the pressure of the first space.

8. A milking apparatus according to claim 1, wherein the partition film is a thin-film-like elastic member.

9. A milking apparatus according to claim 1, wherein the partition film is a hollow expansion/contraction member equipped with a bellows portion.

10. A milking apparatus according to claim 1, wherein provided between the first space and the bottle is a check valve which allows the air in the first space to flow into the bottle and which prevents the air in the bottle from flowing into the first space.

11. A milking apparatus comprising: a milking cup capable of generating a negative pressure inside; and a pad formed of an elastic material and attached to an opening of the milking cup, wherein the pad is provided with a thick-walled portion which is formed more thick-walled than the rest of the pad and which protrudes on the side thereof opposite to the breast when left alone and comes into contact with an inner peripheral surface of the milking cup upon generation of the negative pressure inside the milking cup to thereby protrude on the breast side.
